SEQUENCE LISTING (5 sequences)

SEQ ID NO: 1 - DNA, 3486 bp, Avian infectious bronchitis virus
atgtcggtactattactgttaggactgactcttttgtctgctttatgtgagtgtagtatagtaggtgacaattacacttactattaccacagcgaatatagacctcctaatggttggcatcaacaaggcggagcttatcgtgttattaatactacttcccttagtgtgtctacttgttctttaggtggtgtacctcctctactaactgtaaatgcaccatctgttactatagtagcacccactaagttgccttggtctaaagctaattttacgacagctttttgccattttggtaatttttctgtgtttgtaactcattgtactactggcaactctcggaattgtacattaacaccacctattttagcaggttatattgtagttggtgctaatggttcaattacatataattacacactaagagcgattggtgatttttcacactttcattcttttcagtgtgttagtaattttacttctgtgtacttaaatgggaaactagtttttacgtctaacctttcacttggtgttttaagtgtaggtgcatgtttaaatgcgactggcgcagagctcaagttttctgagtattttaatgtcttagcttactttataaatggtacagcacatgatgttattttatgcgatgatagccctaagggggttttggcttgccagtataatactggtaattttacagatggtttttatccaatttctgttagtactaaggctgatgatgtttttaatgtctatagaactaactctgttttaagttatggttacttacgtaacattacactaagtaattgtactgatgttaaacctaatttagggggtgaactaacactctgtcaacaatatagtgtttcggaaggttatgttaattttaatttttcttttttggatagttttacatatgtaggaccagatctttataattttgactatggttctagtcattcaaattgtcattttacacctaatactattaataatcggttgtcgtttaatactattagtgttgctataggttatggtccaatagatggtgcctgtaagcaaattagatttaggaatggttttaacccatctcatacttgctgttatgcttacagacacccatccttaagtcatagttgtcatggagttactacaggtaagtatagtaatagttatgagtgtattcttatggttgttattagtagggattatgggtctcgtgtgcagacacaaacagaggtacctactagtttaacttatttttataataatattactttggatgtgtgtgttgattataatatttatggtacattaggccaaggttatgttactaatataactgatatggccttatccggtggttacttgggttttggtggattagtcatcctagactcgtctggagctattgacatttttcttgtagaaagtgaaaatggtcctaattattataaaattaatccatgcaatgatgtcaatcagcaatatgtagtgtcagggggtagtatagttggactcttaacatctaataatttaactggttctgaattattagaagatcagttttatgttaaattcactaataatgttcataggcgtagaaggtctattagtgaaaatgttacatcttgtccatatccaacttatggaaagttttgtattaaaccagatggtagtgttagcactatagtgccagaagaagttaaagactatacttctcttttgcttaataggactgattatgtgctcataccagacagttttaatttaactgttacagatgagttcatacagacacaaatgcagcaggttcagattaattgcattcagtatgtgtgtggtaattctatacaatgcaagcaacttttccgacagtatggtcctgtatgtgatagcatactttctatagttaatggcatagtacaacaagataatgctgaaatactccagttttactcttctactaagcctaaagggtttgatgaaagctcttttagtaattttacagctggtgagtttaatatttcgcttgtattacctaagaatggacgacccacagggcgttcgtttatagaggacttgttgtttgacaaagtagaatctttaggactacctggtgactcaacttaccaaaaatgtacgtctggccccctaggttttgttaaagatctagtgtgtgcgcagaagtataatggattacttgtgttgccacctattataacggctgaaatgcaagcaatgtatactagttcattagtagcctcaatggcttttggtgggataacagctgcaggagcaataccttttgcaactcaattgcaagcacgtataaaccatcttggtattactcagacggttttgcaaaagaatcaggagaaaattgctgcatcatttaataaagctatggtgcatatgcaggatggatttaaagccacatctcttgcattacaacaagtgcaagatgttgttaacgagcatggtgcaatacttcagcagactatgcattcacttaataagaattttggtgccataacacatgtcatccaagatatatataaacaactagatgcattagaagcaaatgcacaagttgatcgtttaattaatgggagactttcgtctctatctgtattagcttcttctaaacagttagagtatacaaaagtttcgcatcaacgcgaattagctaaagaaaagattaatgagtgtgtaaaatcacaatctagcaggtatggcttctgtggtgatggcatgcatataatgtccatcccccagagtgcacctaatgggattgtgttcttgcatttcacctacacacctgtatcctttcaaaatgtgacagcagttgtgggtttttgtgttaaacctgttaatggtagtgaatacggccttgtgcctgttgttggtagaggtatttttatagaagtaaatggtacatactatatcacttctcgtgatatgtacaacccaagagctattacagggggtgatgtagttaaactggtttcgtgtcaagctaactatcaaagtattaatagaactgtcattactacatttgtagatgaagatgactttaattttgaccatgagttgtctaagtggtggaatgaaactagccgtgattttccagatctcgaccagtttaactacaccattcctgtactaaatattagtaatgagattgataggattcaagaagttatacagggacttaatgactctctaatagaccttgaaacactatcaatactcaaaacttatattaagtggccttggtatgtttggcttgccatattctttgccattattatctttatccttatactaggatgggttttcttcatgactggatgttgtggttgttgttgtgggtgctttggcattattcctctaatgagtaagtgtggtaagaaatcttcttactacactacttttgataatgatgtggtaactgaacaatacagacctaaaaagtctgtttaa

SEQ ID NO: 2 - protein, 1161 aa, Avian infectious bronchitis virus
MSVLLLLGLTLLSALCECSIVGDNYTYYYHSEYRPPNGWHQQGGAYRVINTTSLSVSTCSLGGVPPLLTVNAPSVTIVAPTKLPWSKANFTTAFCHFGNFSVFVTHCTTGNSRNCTLTPPILAGYIVVGANGSITYNYTLRAIGDFSHFHSFQCVSNFTSVYLNGKLVFTSNLSLGVLSVGACLNATGAELKFSEYFNVLAYFINGTAHDVILCDDSPKGVLACQYNTGNFTDGFYPISVSTKADDVFNVYRTNSVLSYGYLRNITLSNCTDVKPNLGGELTLCQQYSVSEGYVNFNFSFLDSFTYVGPDLYNFDYGSSHSNCHFTPNTINNRLSFNTISVAIGYGPIDGACKQIRFRNGFNPSHTCCYAYRHPSLSHSCHGVTTGKYSNSYECILMVVISRDYGSRVQTQTEVPTSLTYFYNNITLDVCVDYNIYGTLGQGYVTNITDMALSGGYLGFGGLVILDSSGAIDIFLVESENGPNYYKINPCNDVNQQYVVSGGSIVGLLTSNNLTGSELLEDQFYVKFTNNVHRRRRSISENVTSCPYPTYGKFCIKPDGSVSTIVPEEVKDYTSLLLNRTDYVLIPDSFNLTVTDEFIQTQMQQVQINCIQYVCGNSIQCKQLFRQYGPVCDSILSIVNGIVQQDNAEILQFYSSTKPKGFDESSFSNFTAGEFNISLVLPKNGRPTGRSFIEDLLFDKVESLGLPGDSTYQKCTSGPLGFVKDLVCAQKYNGLLVLPPIITAEMQAMYTSSLVASMAFGGITAAGAIPFATQLQARINHLGITQTVLQKNQEKIAASFNKAMVHMQDGFKATSLALQQVQDVVNEHGAILQQTMHSLNKNFGAITHVIQDIYKQLDALEANAQVDRLINGRLSSLSVLASSKQLEYTKVSHQRELAKEKINECVKSQSSRYGFCGDGMHIMSIPQSAPNGIVFLHFTYTPVSFQNVTAVVGFCVKPVNGSEYGLVPVVGRGIFIEVNGTYYITSRDMYNPRAITGGDVVKLVSCQANYQSINRTVITTFVDEDDFNFDHELSKWWNETSRDFPDLDQFNYTIPVLNISNEIDRIQEVIQGLNDSLIDLETLSILKTYIKWPWYVWLAIFFAIIIFILILGWVFFMTGCCGCCCGCFGIIPLMSKCGKKSSYYTTFDNDVVTEQYRPKKSV

SEQ ID NO: 3 - DNA, 23 bp, Artificial Sequence
  Forward Primer for IB80 Real-Time RT-PCR
agtgtagtatagtaggtgacaat

SEQ ID NO: 4 - DNA, 20 bp, Artificial Sequence
  Reverse Primer for IB80 Real-Time RT-PCR
acatcatgtgctgtaccatt

SEQ ID NO: 5 - DNA, 33 bp, Artificial Sequence
  Probe for IB80 Real-Time RT-PCR
ccacctattttagcaggttatattgtagttggt

(19) United States
(12) Patent Application Publication
(10) Pub. No.: US 2019/0276903 A1
(43) Pub. Date: Sep. 12, 2019

(54) METHOD OF DETECTING A NEW VARIANT OF THE IB VIRUS AND KIT THEREFOR

(71) Applicant: ANICON LABOR GMBH, Höltinghausen (DE)

(72) Inventors: Martin LIMAN, Hatten (DE); Theresa OLDOPP, Herßum (DE); Jennifer HANEKE, Cloppenburg (DE); Wiebke BIELENBERG, Syke (DE); Natalie VOGEL, Diepholz (DE); Swaantje RÖNCHEN, Vechta (DE); Diana PETZOLDT, Cloppenburg (DE); Klaus-Peter BEHR, Großenkneten (DE)

(21) Appl. No.: 16/324,724

(22) PCT Filed: Aug. 14, 2017
(86) PCT No.: PCT/EP2017/070618

§ 371 Date: Feb. 11, 2019

(30) Foreign Application Priority Data
Aug. 12, 2016 (DE) 10 2016 215 128.2

(51) Int. Cl. C12Q 1/70

(52) U.S. Cl. CPC C12Q 1/701; C12Q 2600/156

(57) ABSTRACT

The invention concerns a method for detecting the IB virus, variant IB 80, comprising the steps,
a) Providing a sample which potentially contains the IB virus, variant IB 80,
b) Providing a detection system which detects the S gene of the IB virus, variant IB80 with the nucleic acid of the sequence SEQ ID NO: 1 or the product of the S gene of the IB virus, strain IB 80 with the nucleic acid of the sequence SEQ ID No:1 and/or a protein with the amino acid sequence SEQ ID NO: 2, and
c) Detecting of the IB virus, strain IB80 with the detection system in step b) provided in case the provided sample in step a) contains IB virus, strain IB80.

The invention concerns a method to detect a new strain of the IB virus, where the S-protein coding nucleic acid sequence or the amino acid sequence or parts of it is detected. The invention concerns also a kit for such a method.

### BACKGROUND OF THE INVENTION

Throughout the last years, frequent laying performance slumps of chicken herds were observed, which could not be or only partially reduced with common health-promoting measures. After extensive observation, the suspicion arose that this laying performance slump was caused by a virus, a virus that causes the infectious bronchitis.

The infectious bronchitis (IB) is a bird virus disease, which affects especially the common domestic fowl and the pheasant. The pathogen is the Infectious-Bronchitis-Virus (IBV), a corona virus.

Viruses are infectious particles, which are spread as virions outside of the cells (extracellular), but can only replicate themselves as viruses inside (intracellular) a suitable host cell. All viruses contain the genetic information for their reproduction and proliferation, but no ability for self-reproduction or an own metabolism and are therefore dependent on the metabolism of a host cell. Therefore, virologists broadly agree on not counting viruses to the living beings. At the moment, there are 3.000 virus strains identified. Viruses infect cells of eukaryotes (plants, fungi, animals, humans) and prokaryotes (bacteria and archaea).

The corona virus is a member of the Coronaviridae family, which is a family inside the system Nidovirales. The family Coronaviridae is separated on the basis of the phylogenetic characteristics and host spectrum into two sub families and the species Alpha-, Beta- and Gamma- (formerly group 1, 2 and 3 of the old species Coronavirus) and Deltacoronavirus as well as Torovirus and Bafinovirus. The corona virus is assigned to the sub-family Coronaviridae, therein assigned to the species Gamma-Coronavirus. The strain-designation according to ICTV is: avian corona virus. This comprises beneath the pathogen of the infectious-bronchitis virus (IBV), the cause of the infectious bronchitis, also the turkey corona virus (TCoV) and the pheasant corona virus (PHCoV), the duck corona virus, the goose corona virus and the pigeon corona virus.

With genome lengths of more than 30.000 nucleotides, the Coronaviridae belong to the RNA viruses with the largest known genomes. Contrary to the generally large error rate of RNA-polymerases of other RNA viruses, which leads to a limit of genome lengths of circa 10.000 nucleotides, a relatively high genetic stability in terms of the corona virus is reached, i.a. by a 3′ to 5′-exoribonuclease-function of the protein Nsp-14. The single-stranded RNA-genome of the corona viruses is around 27.600 to 31.000 nucleotides (nt) long, with which the corona viruses possess the largest genomes of all known RNA viruses. At the 5′-end a 5′-cap structure and an untranslated region (UTR) of 200 to 400 nt is located, which contains a 65 to 98 nt short, so-called leader sequence. At the 3′-end another UTR with 200 to 500 nt is located which ends in a poly(A) tail. The genome of corona viruses contains 6 to 4 open reading frames (ORFs), of which the two largest, the genes for the non-structural proteins 1a and 1b, lay close to the 5′-tail and overlap each other slightly with different reading patterns. The overlapping position forms a hairpin structure, which allows in 20% to 30% of the reading cycles a reading pattern jump during the translation at the ribosomes and therefore results in a synthesis of smaller amounts of protein 1b.

From the morphologic point of view, the 120 to 160 nm sized virions have a viral envelope, in which 3 to 4 different membrane proteins are embedded: The large, glycosylated S-Protein (180 to 220 kDa) forms as trimer with its club-shaped, about 20 nm to the outside protruding Spikes (Peplomere) the characteristic, crown-shaped look of the corona viruses (lat. Corona: crown, wreath). The membrane protein E (9 to 12 kDa) is embedded in smaller amounts. The human corona virus OC43 and the group 2 corona viruses additionally express the HE-protein (haemagglutinin-esterase-protein, 65 kDa). The also in the evelope embedded M-Protein (23 to 35 kDa) is oriented to the inside and lines the inside of the virus envelope (matrix protein). The inside comprises a helical nucleoprotein complex. This consists of the nucleoprotein N (50 to 60 kDa), which is complexed with a strand of single-stranded RNA with positive polarity. Certain to the outside protruding amino acid residues of the N-protein are interacting with the matrix protein M, so that the capsid is associated with the membrane inner sider.

The representatives of the virus family Coronaviridae cause highly different diseases in various vertebrates such as mammals, rodents, fishes and birds. Corona viruses are genetically highly variable and single virus species are able to infect multiple host strains by overcoming the species barrier.

In birds, the virus is excreted as secretes and excretes. Entries of the virus are especially eyelid conjunctiva and the mucosa of the upper digestive and respiratory tract. The transmission happens as droplet infection, where dust particles and droplets loaded with viruses can travel long distances. The virus colonises the ciliated epithelium of the respiratory tract, but also the reproduction tract and the kidneys can be colonized. The infectious bronchitis spreads rapidly in poultry stocks and especially young stocks show a high morbidity (up to 100%). The mortality varies depending on the virus variant. The incubation time is 18 to 36 hours. The clinical symptoms are shortness of breath, nasal discharge, rattling breathing sounds, sneezing and conjunctivitis. Furthermore, general disorders such as loss of appetite can be observed. Tubal infections lead to laying disorders such as pale, thin-shelled, deformed eggs, wind eggs, significantly diminished or missing laying activity (wrong layers) and diminished hatching rate. The disease duration varies. In some virus strains, a kidney infection can follow, which leads to a high mortality due to kidney failure or sepsis (toxaemia). With superficial respiratory problems, the chickens die due to tracheal occlusion with mucus and other inflammation products.

The fast spreading in the stock and the clinical appearance allow a suspected diagnosis. The diagnosis can be made with the aid of pathological examination of perished birds all well as molecular biological, serological and virological detection methods. Under a differential diagnostics point of view, it is mandatory to differentiate from mycoplasmosis, infectious laryngotracheitis, infectious chicken flu and Newcastle disease as well as from non-infectious diseases (feeding failures, management failures). A treatment is only possible based on the symptoms. The control is therefore based primary on the prophylactic vaccination, which can be done directly after the chickens' hatch (broiler) or in the first life weeks of the animal. Depending on the infection pressure, it is mandatory to repeat the vaccination in endangered areas frequently according to the manufacturers' details and if necessary using multiple vaccine variants.

Due to frequently appearing infections of the infectious bronchitis also in vaccinated stocks at the moment, it seems as if the control- and prophylaxes strategies are improvable. The housing conditions of poultry in the growth and production phase are nevertheless challenging due to the occurrence of the infectious bronchitis. The infection of a stock is of great importance in terms of animal health and the general physical comfort of the infected animals as well as in economic aspects. As mentioned in the beginning, the infectious bronchitis (IB) is an acute and highly infectious disease of the chicken respiratory tract. The disease is caused by the infectious bronchitis virus (IBV), a corona virus, and is characterized by respiratory symptoms, including gasping, coughing, sneezing, tracheal rattle and nasal discharge. In young chicken, severe shortage of breath can occur. In laying hens, shortage of breath, nephritis, (significant) decrease in egg production and the loss of egg quality in the inside (aqueous egg white) as well as at the outside (thin-shelled eggs, soft, irregular, rough shells, missing shells (wind eggs)) can be observed.

Regarding the etiology of the infectious bronchitis virus (IBV), it is important to note that the IBV is the first described corona virus and varies genetically and phenotypically greatly, with hundreds of described serotypes and strains. Corona viruses contain the largest known viral RNA genome in number of nucleotides with nearly 30.000 bases. The RNA forms a single strand and a single segment. The IBV diversity is based on a transcription error, which is highly relevant, if it appears in genomic sequences, which code for proteins, which are part of the adherence to the target cell or of the induction of immune responses. Based on transcription errors, variants can appear, which have an evolutionary advance in disease predisposed chickens. Large genomic changes can happen, e.g. with a complete gene exchange, trough reassortment during replication, where multiple sub-genomic mRNAs are generated and allow the reassortment during coinfections.

The present IBV problematic can be summarized as following:

The infectious bronchitis virus (IBV) is the pathogen of an acute and highly infectious disease which infects chickens of every age and presents a great economic burden in the poultry industry. The virus shows a broad spectrum of anti-genetic and genetic different virus types, which makes the prevention and control of this pathogen extremely complex. The IB virus is mainly present in the chicken. Furthermore, the IB virus or IB virus similar or other bird corona viruses can be present in house- and wild animals, including domestic poultry, partridges, gooses, pigeons, guinea hens, teals, ducks and peacocks (Cavanagh, D., 2005, Coronaviruses in poultry 15 and other birds, Avian Pathol. 34 (6), 439-448; Cavanagh, D., 2007, Coronavirus avian infection bronchitis virus, Vet. Res. 38(2), 281-297).

The IBV is a single-stranded, positive-oriented RNA-virus of the family Coronaviridae, species Gamma corona virus (Cavanagh und Naqi, 2003; International Committee on Taxonomy of viruses, http://www.ictvonline.org/virustaxonomy.asp). The viral genome includes two non-translated regions (UTRs) at the 5′- and 3′-end (Boursnell et al, 1987; Ziebuhr et al, 2000), two overlapping open reading frames (ORFs), which code for the polyproteins 1a and 1b, and regions, which code the most for important structure proteins—Spike (S), Envelope (E), membrane (M) and nucelocapside (N) (Spaan et al, 1988; Sutou et al., 1988.). Furthermore, two accessory genes, ORF3 and ORF5, which express the proteins 3a and 3b or rather the proteins 5a and 5b, are described (Casais et al, 2005; Hodgson et al, 2006; Lai und Cavanagh, 1997). The S-protein (˜3462 nt), which is located in the surface of the virus membrane, is the most important trigger for neutralizing antibodies (Cavanagh und Naqi, 1997; Winter et al., 2008) and is responsible for the virus binding and the penetration into host cells (Cavanagh et al, 1986a; Koch et al, 1990; Niesters et al, 1987). It is split post-translational at a split-position with multiple basic amino acids (Cavanagh et al., 1986b) into the amino-terminal S1-(−535 amino acids) and the carboxyl-terminal S2-sub unit (−627).

The observation that IB serotypes are differing on the genomic level from 20% to 25% and in up to 50% of the amino acids (Cavanagh et al., 2005) of the S1-protein arose significant interest (Cavanagh and Gelb 2008). Such variability can lead to important biological differences between two strains and as a result from a limited amount of amino acid exchanges in the spike-protein, new serotype variations can emerge. Nucleotide heterogeneity is widely spread in the S1-part of the S-gene, and is mainly present in three hypervariable regions (HVRS) in the S1-gene (aa 38-67, 91-141 and 273-387) (Cavanagh et al, 1988; mainly Moore et al., 1997). Correspondingly, the analysis of the full or partly S1-gene-nucleotide sequence is taken to determine the viral genetic type.

At the moment, there are more than 50 different antigenetic and genetic types of the IBV acknowledged, some with remarkable economic effects on the animal husbandry, and some are limited on special geographical regions (de Wit et al, 2011a; 15 Jackwood, 2012). An effective surveillance is primarily relied on the identification of the pathogenic virus type (Jackwood and de Wit, 2013). A variety of methods was developed to differentiate IBV strains. Systems, which examine the antigenetic or genetic properties, allow the description of serotypes and genotypes, whereas methods, which are targeted at the immune response of chickens against a challenge with an IBV-strain, lead to the definition of protector types (Lohr, 1988). Of great importance is otherwise, that the genotype-, serotype- and protector type-based approaches do not group the IB viruses in the same manner. Due to a lack of fast and suited biological assays to classify IBV, analyses of S1-sequence data are the mainly used methods to assign IBV-strains to groups, which were apparently randomly sorted in genetic types, genotypes, classes or clusters.

The infectious bronchitis virus (IBV) is the pathogen of a highly infectious disease in the global poultry farming, which causes severe economical losses. The virus exists as a variety of genetical different virus types. Phylogenetic analysis as well as measurements of the pair-wise similarity between nucleotide- or amino acid sequences were used to classify IBV strains. Until recently, there was no consensus about the method, with which the IBV sequences should be compared. Heterogenic terms of genetical groups, which are incompatible with the phylogenetic history were accepted, which results in a confusing coexistence of various systems of genotyping.

The acceptance of an international accepted viral nomenclature is nevertheless of significant importance for the conduction of profound studies and to obtain and evaluate new findings regarding epidemiology and evolution of the IBV. To resolve the above described problematic state of science, Valastro et al., developed a, published in 2016 (Valastro V., Holmes E. C., Britton P., Fusaro A., Jackwood M. W., Cattoli G., Monne I., S1 gene-based phylogeny of infectious bronchitis virus: An attempt to harmonize virus classification, Infection, Genetics and Evolution, 10 39 (2016), 349-364), new classification scheme on the basis of phylogenetically relationships (based on the S1-gene), which can be updated and reworked, as soon as new S1-sequences are available. Valastro et al. describe a simple and reproducible phylogenic-based classification system combined with a defined and rational nomenclature of the line of ancestry for the assignment of IBV strains. With the usage of complete S1 gene sequences, these could be compared with each other and percentage similarities and divergences to each other could be determined. The sequence analysis resulted in a classification into 6 genotypes, wherein a threshold of about 30% sequence difference was determined between the single IBV strains in the S1 gene, at which point the strains were classified to a new genotype. The large majority of the IBV strains belong to the genotype I and forms sub clusters inside this genotype, which are called lines inside the genotype. For the genotype I 27 lines and for the genotypes II-VI one line each were described. Beneath the genotypes and lines, single S1 gene sequences of IBV strains are describe as UV (unique variant). These could not be classified to the defined lines. Due to the extensive change rate within the IB viruses, Vastro et al. propose that the phylogenetic relationships are a better suited criterion for the sequence classification instead of pair-wise sequence comparisons, especially that the proposed by Valastro et al. classification scheme can be updated and reworked if new S1 sequences were found.

The inventors achieved to isolate a new variant of the IBV and to sequence it. This new variant and also its variants were named also IB80 below. The IB variant “IB 80” is further described in the German patent application with the application number DE 102016215118.5. By way of reference, this application is encorporated herein especially in terms of the herein revealed sequences. This new virus is made jointly responsible for the described laying performance slumps.

Accordingly, there is a need to have an appropriate detection method for the presence of the IB variant “IB 80” in a biological sample provided. Preferably, this method should consist of a high specificity and especially can distinguish between other IBV strains.

### DESCRIPTION OF THE INVENTION

This task is solved by a method to detect the IB virus, variant IB80, comprising the steps,

a) providing a sample which potentially contains the IB virus, variant IB80,
b) providing a detection system which detects the S gene of the IB virus, strain IB80 with the nucleic acid of the sequence SEQ ID NO: 1 or the product of the S gene of the IB virus, variant IB80 with the nucleic acid of the sequence SEQ ID NO: 1 and/or detects a protein with the amino acid sequence SEQID NO: 2, and
c) detecting of the IB virus, variant IB80 with the in step b) provided detection system in case that the provided sample in step a) contains IB virus, variant IB 80.

Key of the method is herein, that the detection system is based on the detection of the presence of the S gene of the IB80 virus or of the gene product of the S gene. The sequence was not known so far and the finding of this sequence allows firstly a detection method.

As detection method based on the previous described, all for this purpose suited detection systems, which are generally recognized from a scientific point of view are appropriate. Preferred examples for such detection methods are immunofluorescence based methods, detection per isothermal amplification, real-time PCR detection with dyes such as SYBR, real-time PCR based detection with Tagman probes, molecular beacons, minor groover binder or ampliflour, fluorescence resonance energy transfer (FRET), fluorescence in situ hybridisation, antibody based detection methods such as ELISA or lateral flow test systems and especially a real-time RT-PCR. As sample, principally every sample is suited, which is suited for the corresponding detection method. The person skilled in the art will choose the according selection.

In step b) of the method according to the invention, a detection system is provided, which can detect the S gene of the IB80 virus or the gene product of this S gene. Herein it is self-evident sufficient for the detection, that also the presence of fragments of this gene or rather of the protein can be detected. Preferably, the detection system is so specific that it can detect sequences with a sequence ID identity to SEQ ID: NO: 1 of ≥85% or rather to the amino acid sequence SEQ ID NO: 2 of also ≥85%. Preferably, the detection according to the invention affects only sequences with an identity of ≥90%, moreover preferred of ≥95%, furthermore preferred of ≥98% and especially preferred of ≥99% to SEQ ID NO: 1 or NO: 2.

Preferred is a method according to the invention, wherein the in step b) provided system comprises specific antibodies against a nucleic acid of the sequence SEQ ID NO: 1 or of the product of the S gene of the OB virus, variant IB80 with the nucleic acid of the sequence SEQ ID NO: 1 and/or a protein with the amino acid sequence SEQ ID No: 2. Due to the resolved genetical structure of the IB80-virus, it is possible to generate matching antibodies, preferred monoclonal antibodies, against the nucleic acid or the S gene or rather the amino acid sequence of the corresponding protein. For this purpose, it is referred to the above mentioned German patent application. To the skilled person, there are known a variety of antibody based detection methods. Therefore, it is possible to use a reliable detection method based on antibodies.

Preferred is a method according to the invention, wherein the system provided in step b) ensures a reverse transcription of the nucleic acid sequence SEQ ID NO: 1 or a part of it into DNA.

As IB-viruses are RNA viruses, it can be beneficial for detection methods, that the RNA of these viruses, especially the sequence of the S1 gene or a part of it can be transcribed into DNA.

Preferred, the reverse transcription happens due to a M-MLV reverse transcriptase or similar.

Preferred is a method according to the invention, wherein the system provided in step b) ensures an amplification of at least one section of the into DNA transcribed nucleic acid of the sequence SEQ ID NO: 1. Detection systems, which are based on the amplification of DNA are well known in the art.

Preferred is that the amplification is made by the use of Taq DNA polymerase or similar. Especially preferred is a method according to the invention, wherein the system provided in step b) comprises of a probe, which generates a detection signal under PCR conditions, against a section of the into DNA transcribed nucleic acid of the sequence SEQ ID NO: 1.

In this context “under PCR conditions” means that the probe binds to a section of the transcribed nucleic acid especially an amplified section of the transcribed nucleic acid during the amplification phase trough the polymerase chain reaction (PCR).

The detection signal of the probe can be every suited detection signal, for example a dye, radioactive labelling, wherein a fluorescence signal is particularly preferred in the sense of the invention.

With these signals and the binding ability of the probe, it is possible, to gain findings regarding the presence of IB80 and also regarding the concentration of this variant in the measured sample, already in the amplification phase.

Preferred is a method according to the invention, wherein the detection system comprises a real-time RT-PCR.

Real-Time PCR is a method, which is established for various applications. It delivers fast and reliable data concerning the compound which should be detected, in this case the virus variant IB80.

Preferred is a method according to the invention, wherein the detection system comprises a primer of the SEQ ID NO: 3 and/or primer of the SEQ ID NO: 4 and/or a probe of the sequence SEQ ID NO: 5.

Especially preferred in this context is that a primer of the SEQ ID NO: 3 and a primer of the SEQ ID NO: 4 is used. It is particularly preferred that to this use a probe of the SEQ ID NO: 5 is used additionally.

These primers and these probes have turned out to be especially suitable for the method according to the invention.

Preferred is a method according to the invention, wherein the detection system comprises a positive control for the IB virus, strain IB80 and/or a negative control for the IB virus, strain IB80.

When a positive and negative control is set, the results of the detection method are especially well verifiable.

Part of the invention is also a kit for a method according to the invention, including a primer pair for the amplification of a section of into DNA transcribed nucleic acid of the sequence SEQ ID NO. 1 or specific antibodies against (i) a nucleic acid of the sequence SEQ ID NO: 1 and/or (ii) the product of the S gene of the IB virus, variant IB80 with the nucleic acid of the sequence SEQ ID NO: 1 and/or a protein with the amino acid sequence SEQ ID NO: 2 as well as more reagents for the detection method as described above.

Preferred is that the kit according to the invention comprises a primer pair according to SEQ ID NO: 3 and SEQ ID NO: 4.

With this kit, the method according to the invention is very well conductible.

Preferred is a kit according to the invention, which comprises as further reagents a probe to bind a section of the in SNA transcribed nucleic acid of the sequence SEQ ID NO: 1 and/or one or more suited buffers and/or suited nucleotides and/or a RNA dependent DNA polymerase and/or a DNA dependent DNA polymerase to amplify a section of the in DNA transcribed nucleic acid of the sequence SEQ ID NO: 1.

Thereby, the reagents are preferred the above described variants.

### Explanation of the Sequences:

SEQ ID NO: 1 S gene of IB80
SEQ ID NO: 2 S protein of IB80
SEQ ID NO: 3 Example of a forward primer
SEQ ID NO: 4 Example of a reverse primer
SEQ ID NO: 5 Example of a probe

Hereinafter, the invention is defined further using an example:

### Example

System for a method according to the invention:

### 1. Oligonucleotide Design:

Based on the genome sequence of the S gene (SEQ ID NO:1) of the infectious bronchitis virus “strain IB80”, sequences for primer pairs as well as according hydrolyse probes for the specific detection in the real-time RT-PCR were worked out manually and were established. Due to the high divergence to all other IBV-strains in the gene sequence of this region, the S1 gene was selected for the detection per real-time PCR. A preferred primer/probe setup for a real-time PCR is depicted in table 1.

TABLE 1
Primer-and special sequences IB80 Real-Time RT-PCR

| | | SEQ ID |
|---|---|---|
| Name | Sequence 5′-3′ | NO: |
| IB80-F | AGTGTAGTATAGTAGGTGACAAT | 3 |
| IB80-R | ACATCATGTGCTGTACCATT | 4 |
| IB80-P | FAM-CCACCTATTTTAGCAGGTTATATTGTAGTTGGT-BHQ1 | 5 |

### 2. Real-Time RT-PCR Setup

The setup for the real-time RT-PCR with a final volume of 20 μL is listed in table 2.

TABLE 2
PCR Setup

| Component | Volume | Final concentration |
|---|---|---|
| BCD 2x RT-qPCR-Mix | 10 μL | 1x |
| (AniCon Labor GmbH, | | |
| Germany) | | |
| IB80-F | var | 400 nM |
| IB80-R | var | 400 nM |
| IB80-P | var | 100 nM |
| H₂O molbiol | Add 16 μL | |
| RNA (sample) | 4 μl | |

### 3. Temperature Profile

The IB80 real-time RT-PCR is conducted under usage of the BCD 2×RT-qPCR mix, according to the temperature profile listed in table 3.

TABLE 3
temperature profile of the IB80 real-time RT-PCR

| Step | Function | Temperature/Time | Cycles |
|---|---|---|---|
| 1 | Reverse | 50° C. for 10 min | 1x |
| | transcription | | |
| 2 | Activation | 95° C. for 1 min | 1x |
| | polymerase and | | |
| | denaturation | | |
| 3 | Denaturation | 95° C. for 10 sec | 42 repetitions in |
| 4 | Annealing and | 60° C. for 60 sec | total of step 3 & 4 |
| | Extension | | |

### 4. Controls

To secure the validity of the real-time RT-PCR run, an IB80 positive control (virus isolate stored at Public Health England—National Collection of Pathogenic Viruses) and/or a negative control can be used. The IB80 positive control should be determined on a CT-value between 25 and 30, to secure a stable amplification at a low cross-contamination risk.

The quality of the RNA preparation of the questionable sample regarding the suitability of the reverse transcription and amplification should be examined preferentially, e.g. using a parallel detection of so called “housekeeping” target genes or RNA products added artificially to the sample.

### 5. Evaluation

All samples with a CT value <42 in the FAM specific canal are evaluate as positive. Only curve characteristics with a typical exponential amplification are seen as positive. With the system of this example, the method according to the invention can be performed reliably and the IB80 can be detected in biological samples.

1. A method for detecting the IB 80 variant of the Infectious Bronchitis (IB) virus, comprising the steps,
a) providing a sample which potentially contains the IB 80 variant,
b) providing a detection system which detects the S gene of the IB80 variant, the S gene having the nucleic acid sequence set forth as SEQ ID NO: 1, and/or which detects the product of the S gene of the IB 80 variant, the S gene having the nucleic acid sequence set forth as SEQ ID No:1 and/or which detects a protein having the amino acid sequence set forth as SEQ ID NO: 2 and
c) detecting the IB 80 variant with the detection system in step b) if the provided sample in step a) contains the IB 80 variant.

2. The method according to claim 1, wherein the system provided in step b) comprises specific antibodies against a nucleic acid having the sequence set forth as SEQ ID NO: 1 and/or comprises specific antibodies against the product of the S gene of the IB 80 variant, the S gene having the nucleic acid sequence set forth as SEQ ID NO: 1 and/or comprises specific antibodies against a protein having the amino acid sequence set forth as SEQ ID NO: 2.

3. The method according to claim 1, wherein the system provided in step b) ensures a reverse transcription of the nucleic acid sequence set forth as SEQ ID NO: 1 or a part thereof into DNA.

4. The method according to claim 3, wherein the system provided in step b) ensures an amplification of at least one section of the nucleic acid sequence set forth as SEQ ID NO: 1 transcribed into DNA.

5. The method according to claim 3, wherein the system provided in step b) comprises a probe, which generates a detection signal under PCR conditions, against a section of the nucleic acid sequence set forth as SEQ ID NO: 1 transcribed into DNA.

6. The method according to claim 3, wherein the detection system comprises a Real-Time RT-PCR.

7. The method according to claim 3, wherein the detection system comprises a primer having the nucleic acid sequence set forth as SEQ ID NO: 3 and/or a primer having the nucleic acid sequence set forth as SEQ ID NO: 4 and/or a probe having the nucleic acid sequence set forth as SEQ ID NO:5.

8. The method according to claim 1, wherein the detection system comprises a positive control for the IB 80 variant and/or a negative control for the IB 80 variant.

9. A kit for a method according to claim 1, comprising a primer pair for the amplification of a section of the nucleic acid sequence set forth as SEQ ID No: 1 transcribed into DNA or specific antibodies against (i) a nucleic acid having the sequence set forth as SEQ ID NO: 1 and/or (ii) the product of the S-gene of the IB 80 variant, the S-gene having the nucleic acid sequence set forth as SEQ ID No: 1 and/or a protein having the amino acid sequence set forth as SEQ ID NO: 2 and further reagents for the detection method as defined in claim 1.

10. A kit according to claim 9, comprising as further reagents a probe to binds to a section of the nucleic acid having the sequence set forth as SEQ ID NO: 1 transcribed into DNA and/or one or more suitable buffers and/or suitable nucleotides and/or a RNA-dependent DNA-polymerase and/or a DNA-dependent DNA-polymerase to amplify a part of the nucleic acid sequence set forth as SEQ ID NO: 1 transcribed into DNA.

11. The method according to claim 4, wherein the system provided in step b) comprises a probe, which generates a detection signal under PCR conditions, against a section of the nucleic acid sequence set forth as SEQ ID NO: 1 transcribed into DNA.

12. The method according to claim 4, wherein the detection system comprises a Real-Time RT-PCR.

13. The method according to claim 5, wherein the detection system comprises a Real-Time RT-PCR.

14. The method according to claim 4, wherein the detection system comprises a primer having the nucleic acid sequence set forth as SEQ ID NO: 3 and/or a primer having the nucleic acid sequence set forth as SEQ ID NO: 4 and/or a probe having the nucleic acid sequence set forth as SEQ ID NO:5.

15. The method according to claim 5, wherein the detection system comprises a primer having the nucleic acid sequence set forth as SEQ ID NO: 3 and/or a primer having the nucleic acid sequence set forth as SEQ ID NO: 4 and/or a probe having the nucleic acid sequence set forth as SEQ ID NO:5.

16. The method according to claim 6, wherein the detection system comprises a primer having the nucleic acid sequence set forth as SEQ ID NO: 3 and/or a primer having the nucleic acid sequence set forth as SEQ ID NO: 4 and/or a probe having the nucleic acid sequence set forth as SEQ ID NO:5.

17. The method according to claim 2, wherein the detection system comprises a positive control for the IB 80 variant and/or a negative control for the IB 80 variant.

18. The method according to claim 3, wherein the detection system comprises a positive control for the IB 80 variant and/or a negative control for the IB 80 variant.

19. The method according to claim 4, wherein the detection system comprises a positive control for the IB 80 variant and/or a negative control for the IB 80 variant.

20. The method according to claim 5, wherein the detection system comprises a positive control for the IB 80 variant and/or a negative control for the IB 80 variant.
